(19) United States
(12) Patent Application Publication
(10) Pub. No.: US 2002/0004199 A1
(43) Pub. Date: Jan. 10, 2002

(54) No wash bead assay, kit and procedure

(76) Inventor: Mark K. Hechinger, Pasadena, CA (US)

Correspondence Address: COLIN P ABRAHAMS, 5850 CANOGA AVENUE, SUITE 400, WOODLAND HILLS, CA, 91367

(21) Appl. No.: 09/873,866

(22) Filed: Jun. 4, 2001

Related U.S. Application Data
(60) Provisional application No. 60/209,437, filed on Jun. 2, 2000.

(51) Int. Cl. C12Q 1/70; G01N 33/53; G01N 33/537; G01N 33/543

(52) U.S. Cl. 435/5; 435/7.92; 436/518

(57) ABSTRACT

A method of making a no wash bead based assay comprises preparing a first reagent comprising a buffer, and preparing a second reagent comprising a protein. Beads of preselected size and having a coefficient of variation less than 5% are prepared, including washing the beads in the buffer to form a bead-buffer matrix and reducing the surfactancy of the beads to an effective amount. Thereafter, an antigen for detecting the presence of a target species is added to the bead-buffer matrix such that the antigen attaches to the beads to form a bead-antigen mixture. The surfactancy of the beads facilitates attachment of the antigen thereto. Buffer is added to the bead-antigen mixture and thereafter the mixture is incubated. The second reagent is added to the bead-antigen mixture to reduce or eliminate non-specific binding sites.

### CROSS REFERENCE TO RELATED APPLICATIONS

This application claims the benefit of U.S. Provisional Patent Application No. 60/209,437 filed Jun. 2, 2000, which is incorporated herein by reference in its entirety.

### FIELD AND BACKGROUND OF THE INVENTION

This invention relates to assays, assay kits, and the methods of preparation thereof. The assays are all bead-based for use with a flow cytometer, whereby beads of different sizes and/or colors, each having different attachments, may be analyzed using flow cytometry techniques.

Technological advances in bead-based flow cytometric analysis have created an overwhelming demand for fast and efficient methods for the detection and/or identification of antibodies, antigens, enzymes, proteins, chemicals or other substances which will bind to beads or bead systems which have been coated with the appropriate ligand. In this specification (including claims), the terms “ligand” and “antigen” are intended to have a broad meaning, namely, a substance that binds to a complementary substance, unless the context clearly indicates otherwise. An example of such binding between a substance and its complementary substance includes, but is not limited to, an antigen-antibody combination, where both are ligands. Bead-based assay systems utilize laser flow cytometers which may detect the size, forward angle light scatter (FALS), fluorescence and other parameters of beads flowing through the flow cytometer. The beads, or at least a portion thereof, may have attached directly or indirectly thereto antibodies, antigens, enzymes, proteins or the like. The presence and/or characteristics of the beads and/or the substances attached thereto are measured and recorded as the beads, with and without attachments, flow through the flow cytometer.

The parameters and/or physical characteristics of the bead, or the bead when attached to the antibody, enzyme, protein, or other substance, enable one to distinguish unique sizing properties of single or multiple bead systems, while at the same time detecting fluorescent dyes attached to specific antibodies, enzymes etc., which bind to the bead itself.

Some systems only seek to detect and measure agglutination of beads in the reaction, which can also be detected in a flow cytometer.

There are many ways in which these bead-based assay systems can operate. Regardless of the system, however, there is always a constant problem of carrying multiple procedures and steps before the bead with attachment can be analyzed by the flow cytometer. Such steps include the adding of substrate with the beads, washing, adding the detector system (such as, for example, a fluorescent antibody, enzyme or chemical), incubating and then washing once more. Moreover, some methodologies may require even more than two wash steps. As a result, the large number of steps needed in the processing of the assay to some degree defeats the purpose of developing the bead-based flow cytometry system in the first place, the goal of which is to save time and labor.

### SUMMARY OF THE INVENTION

According to one aspect of the invention, there is provided a method of making a no wash bead based assay, the method comprising: preparing a first reagent comprising a buffer; preparing a second reagent comprising a protein; preparing beads of preselected size and having a coefficient of variation less than 5%, including washing the beads in the buffer to form a bead-buffer matrix and reducing the surfactancy of the beads to an effective amount; adding an antigen for detecting the presence of a target species to the bead-buffer matrix such that the antigen attaches to the beads to form a bead-antigen mixture, the surfactancy of the beads facilitating attachment of the antigen thereto; adding buffer to the bead-antigen mixture and thereafter incubating the mixture; and adding second reagent to the bead-antigen mixture to reduce or eliminate non-specific binding sites.

The present invention is therefore directed, in one aspect, to the provision of a bead assay kit and procedures incorporating a “no-wash” capability, thereby saving the operator a considerable amount of time compared to more conventional or classic methodologies. Thus, the invention relates to a process, bead system and/or assay kit prepared so that the end user can test for the presence of a target substance or species simply and in a short time, without having to carry out complicated procedures, including multiple washes, in order to obtain a result.

Certainly, the “no-wash” method and kit of the invention reduces the number of steps and shortens the amount of time needed to conduct standard bead-based flow cytometer assays. The present invention also provides, in another aspect, a method and kit whereby the need to adjust for background may be significantly reduced or even virtually eliminated and the coefficient of variations of multiple bead-based systems is optimized with minimal or no agglutination. The background which may affect the results of any assay may include, for example, fluorescent signals that are non-specific to the assay itself, or may be due to autofluorescence, or non-specific binding of indicator reagents, to the beads.

In one aspect, the present invention describes a kit and procedure for detecting the presence of a target substance or species wherein any bead-based analyte detection system can be used, and incubated in the same reaction vessel. In other words, the multiple steps associated with the assay process are reduced to provide a more efficient testing mechanism in a shorter time. In one form of the invention, the beads may be separately packaged, or they may be combined in a mixed slurry of different sizes and/or colors. As mentioned, multiple washing steps are not needed, reducing the amount of time and effort required to conduct the assay. While conventional bead-based assays are currently replacing, at least in part, the traditional ELISA assay which requires sample pre-dilutions, multiple washes and separate indicators, multiple steps are nevertheless still required by these conventional bead-based assays, making them more cumbersome to package as a kit and more difficult to carry out by the end user.

In a further aspect, the present invention attempts to optimize the conditions for detection and analysis of bead based systems used in flow cytometry apparatus by utilizing “no-wash” procedures throughout the entire assay. The invention addresses, in one aspect, some of the problems in conventional bead staining assay procedures, where attempts at utilizing a “no-wash” methodologies have mostly resulted in beads which tend to agglutinate, which may have doublets and/or have considerable “background” fluorescence. The results obtained in such an assay can therefore be highly subjective since they are difficult to read and lack clarity.

The assay preparation is initiated by choosing the proper bead material, which preferably has a low coefficient of variation (C.V.) for bead size. A lower C.V. is preferable, since the lower C.V. per bead size can result in the utilization of more beads of different sizes or colors in a particular tube or assay. Generally, a low C.V. means that there will be less variation in bead size in a given sample or lot so that all beads in that lot will tend to be of more uniform size. Conversely, a high C.V. is indicative of a higher variation of size, and in this situation, the various sizes of beads selected for use in an assay would have to be such that the beads used differed in size by greater amounts. The bead material chosen may be appropriate for protein coating. The beads are then carefully prewashed (centrifuged/decanted) with a carbonate buffer, and coated with a specific concentration of antigen or other ligand as determined by serial titrations (generally in microgram concentrations) then mixed. Buffer is then added on top of the bead pellet to an appropriate volume. The mixture is allowed to incubate for several minutes or several hours, after which it is centrifuged, and the beads washed with a carbonate buffer. Depending upon the system, the resultant bead/buffer mixture will have a pH of between 5 and 10.

The mixture may be subjected to vortex suspension, and, in one example only, may be coated with a protein or other type of solution, for example 0.1%-10%, according to the system or the requirements of the assay. This solution inhibits the non-specific binding of indicator reagents or sample constituents, leaving more distinct separations between truly positive signals, by reducing the amount of false positive signals which may confuse readings of the assay.

The mixture is then vortexed, spun and decanted. A final carbonate buffer suspension would resuspend the bead pellet to its final concentration after a gentle vortex.

The invention may incorporate the use of positive and negative controls which can be incubated with the bead. Optimizing the total volume ratio of sample to bead suspension in the control, as compared to the reference, may greatly increase the ability of the assay to mimic the results of the predicate device [i.e. ELISA] and help reach higher thresholds of sensitivity. For example, by fixing the bead/carbonate concentration per assay, the sample may need to be pre-diluted before adding to the bead suspension. As in the ELISA assays, prediluting patient samples established an equilibrium between the detection antigen, patient sample and indicator system, thus optimizing sensitivity and specificity.

The invention also envisages the use of indicator material, such as fluorescently labeled antibodies, antigens, enzyme substrates, or other chemical compounds, and these can be added after an appropriate incubation period, for example, an incubation period of approximately fifteen to thirty minutes. Phosphate buffer saline (PBS) may be added, but is not necessary, to constitute the final stopping solution and background eliminator. Preferably, sufficient amounts of PBS should be added to optimize analysis by the flow cytometer. Nevertheless, relative concentrations of beads, antigens, indicator reagents should be optimized prior to use without the addition of PBS.

Fluorescent and scatter photomultipliers (PMT) voltages may also be optimized according to the invention by utilizing a bead incubated with normal or negative staining sample. These electronic detectors adjust the analog signal produced by the beads and their fluorescent properties into digital displays. Optimizing the flow cytometer means, 1) displaying all the beads on a size or fluorescent graph and, 2) adjusting the fluorescent graphs, used for quantitation, on a negative or “normal” control (e.g. no or low signal). Alignment of the fluorescent channels may also encompass the use of standardized fluorescent material (beads) which could potentially set a consistent mean channel value. This would eliminate daily instrument (result) variations.

### DETAILED DESCRIPTION OF THE INVENTION

The invention is for bead-based flow cytometry assays, which allow for the detection of several antibodies, antigens or other ligands, using the same dilution and reaction vessel, and which therefore simplifies the assay, and also decreases the time thereof. The bead based assay is manufactured in a way which allows the assay to proceed so that no (or reduced) amounts of washing of the assay during the testing procedure is required. The use of a single reaction vessel and minimal steps in setting up the assay for analysis by a flow cytometer results in a more efficient and accurate test for the target substance.

In one particular embodiment, the following antigens are attached to beads, each antigen preferably being attached to a bead of a different size and/or color:

RnP/Sm Antigen

Sm Antigen

SS-A Antigen

SS-B Antigen

Scl-70 Antigen

dsDNA Antigen

Beads may also be attached to other components besides antigens, examples of which are as follows:

Histones

Lipids

Viral antibodies

Viral antigens

Bacterial antibodies

Bacterial antigens

Recombinant proteins

Cellular antigens

Other chemicals

Although the particular antigens described above have been selected in this embodiment to assay for the detection of the corresponding antibodies, it will be appreciated that the invention is not limited to detecting such antibodies, and the above list is therefore a representative, but not an exclusive, sample. Further, the invention is not limited to the use of antigens, but may comprise any ligand (antibody/antigen) that can be used in bead based flow cytometry. Furthermore, the detecting system may combine fluorescent beads, and/or secondary fluorescent indicators and/or different sizes of beads.

The invention also requires various beads to which the antigens will be attached, and the following list constitutes a preferred selection of beads of various sizes, combination of sizes and different impregnated colors which have been found to work well in the assay. These beads are as follows:

3μ latex beads

4μ latex beads

5μ latex beads

6μ latex beads

7μ latex beads

8μ latex beads

10μ latex beads.

The above beads have been selected as an embodiment of the present invention based on their size differences, and size spread with respect to each other, as well as their size uniformity within each bead population as providing a workable group for conducting multiple assays in a single procedure. Different combinations of sizes etc. can be used. As such, it will be appreciated that the invention provides for a bead based assay which is capable of detecting multiple target substances in a single test. The bead assay would comprise a mix of a plurality of beads, each attached to an antigen, antibody etc., so that the assay would simultaneously test for the antibody, antigen etc. respectively at one time.

The method for preparing a bead based assay, and manufacturing a kit which includes the bead based assay, therefore enables the end user of the kit to carry out the “no-wash” procedure of the invention. In this specification, the term “no wash” is intended to be broadly interpreted, and to signify the ability of the end user of the bead based kit to test for the presence of a target substance in a procedure which requires fewer steps, especially washing steps, in order to obtain a result more quickly and accurately.

Preparation of the assay and the kit of the invention involves a number of steps, and initially requires the preparation of certain reagents for use in the procedure and testing, as detailed below.

Reagents

Three reagents are preferably required in the preparation of the bead based assay and kit of the invention, as set out below.

The first reagent comprises a carbonate buffer which may be prepared, in one embodiment, by placing one liter of sterile water in a volumetric flask and adding 3 grams of sodium carbonate and 1.6 grams of sodium bicarbonate into the flask of water. The components are thoroughly mixed, producing a carbonate buffer having a pH between about 9 and 10, preferably approximately 9.6, and being within a preferred range of +/−0.1 of pH 9.6. The carbonate buffer prepared as described above may then be stored for approximately one week at a temperature of 2-8° C.

A second reagent which is required in the “no-wash” procedure of the invention is, preferably, Bovine Serum Albumin (BSA), or related a protein, preferably in saline. Preferably, the reagent composition comprises a 0.5% BSA in saline, although effective variations in this concentration may also be used (0.1% to 5%). In the invention, a preferred embodiment requires 100 mLs of this mixture. The reagent is prepared by carefully weighing 1 gram of BSA, and adding it to a sterile 100 mL bottle. Thereafter, 100 mLs of sterile, physiological, saline is added, and mixed with the 1 gram of BSA until the BSA completely dissolves in the saline. The reagent so prepared may thereafter be stored in a refrigerator, and has a shelf life of up to 6 months.

Still another reagent used is the same as above, but the diluent is carbonate buffer used for washing the beads after they have been coated with antigens.

The third reagent prepared in the “no-wash” process is, for example, Goat anti-Human Ig, F(ab′)²⁻ FITC , or another indicator substance which is matched to the analyte, or target substance being tested. This reagent is prepared, in one embodiment of the invention, in a 1:20 ratio. The reagent is prepared by adding to a sterile 100 mL bottle 95 mLs of 0.5% Bovine Serum Albumin (BSA). Using a sterile pipette, 5 mLs of neat or undiluted goat anti-human Ig is added to the bottle, and the components are mixed until evenly distributed. The bottle containing the mixture is preferably wrapped with aluminum foil or stored in an amber bottle protected from light, and can be held stored in a refrigerator. The mixture will have a shelf life of up to six months.

This third reagent is used in the testing stage, as opposed to the assay preparation stage, for the purpose of identifying the target substance being tested, and now attached to the head. The third reagent will be specifically selected so as to enable it to identify the particular target substance whose presence is being detected.

Other fluorescently conjugated antibodies, antigens etc. may also be used within the scope of the invention, and these antibodies and/or antigens may be immunologically dependent upon the assay in development.

For optimal effectiveness in preparing the bead based assay for use in testing so as to have a “no wash” procedure, and to ensure effectiveness of the bead based product, it is preferable that the reagents stored in the refrigerator are, as far as possible, protected or shielded from light, and that repeated removal of the reagents from the refrigerator is avoided. In a preferred form, the neat conjugate is stored frozen. Further, the refrigerated components are best preserved in terms of their effectiveness where more than one freeze cycle is eliminated or avoided. Additionally, the storage should preferably be under sterile conditions.

Procedure for Coating Beads With Antigen

The procedure for coating the beads with antigen, and subsequently carrying out quality control steps, are described in further detail below. It is of course preferred that the procedure for coating the beads with antigen may take place in a certified GMP facility (Good Manufacturing Practice, which comprise laboratories certified by the FDA). Further, while not specifically described, standard operating procedures (SOP's), generally acknowledged by persons skilled in the industry, should also preferably be in place for all quality control techniques in order to ensure the integrity of the assay and the method for their preparation.

It will also be understood that, while the coating of the beads described below may be with extractible nuclear antigens (ENAs), the assay and procedure for its preparation are equally valid with respect to coatings with other antigens or ligands generally, for example, viral recombinant proteins for detecting anti-viral antibodies.

In a preferred form, the bead based assay is manufactured in preselected amounts using typical or conventional lot quantities. Therefore, it is initially important to determine the amount of antigen, beads and conjugate which are necessary to manufacture the desired lot quantity. This is accomplished through several pre-manufacturing titration experiments using multiple concentrations of each of the components. This is known as a “block titration”. Set out below in Table I is an indication of the recommended amount of beads per 1 mL of buffer, where the concentration has been determined. A varied final dilution in carbonate buffer optimizes all the lot components.

TABLE I

| Bead Size | | | | |
|---|---|---|---|---|
| (μ) | % Solids | C.V. | Amt. (uL.)/mL | Test |
| 3 | 0.05 | 1.2% | 250 | Scl-70 |
| 4 | 0.35 | 1.0% | 100 | RnP/Sm |
| 5 | 0.30 | 1.2% | 200 | Sm |
| 6 | 0.30 | 1.2% | 300 | SS-A |
| 7 | 0.29 | 1.3% | 700 | SS-B |
| 8 | 0.28 | 1.2% | 800 | dsDNA |

In the above Table, six bead sizes are provided, ranging from 3 to 8 microns in diameter. A bead of particular size is shown in the Table as being coated with a particular antigen. Thus, the bead of size 3 microns will be coated with antigen Scl-70, the bead of size 4 microns is coated with antigen RnP/Sm, etc. Under the heading “Test” the antigen coated to the particular bead size is set forth.

The data under the heading Percentage (%) Solids represent the gram weight of latex bead in solution per 100 mLs. Under the heading “C.V.”, the coefficient of variation of the bead size is provided. All have a coefficient of variation which ranges between 1.0% and 1.3%. The lower the C.V., the less will be the variation in size of the beads within a particular bead size (see column 1 in the Table above), while the converse would apply as well.

Finally, under the heading “Amount”, the bead concentration in microliters per mL is provided. The bead concentrations indicated in the Table, while preferred and important, can nevertheless be varied according to the circumstances. However, it has been found that the bead concentrations shown above facilitate an assay and procedure which provides improved and possibly more accurate results, as described further below.

Once the optimal amount of antigen, beads and conjugate have been determined depending upon the quantity required, and based on the information contained in Table I above, the beads which have preferably been stored in the refrigerator are removed therefrom and allowed to equilibrate to room temperature before use. Note that “optimal” concentrations are those which produce the highest mean fluorescent intensity (MFI) while minimizing aggregation and background fluorescence.

For manufacturing purposes, a container for each of the beads with antigen coating to be prepared is provided. In a preferred embodiment, there are six containers, each having a 250 mL volume, and they are preferably flat-bottom centrifuge containers. Each one of the six containers is appropriately labeled with one of the six antigens under the heading “Test” in Table I above, namely, RnP/Sm, Sm, SS-A, SS-B, Scl-70, and dsDNA. The predetermined amount of antigen may then be placed in each of the containers, with appropriate labeling.

Thereafter, buffer, as prepared in accordance with the description above under the heading “Reagents”, is added to the centrifuge containers in the amount of 1 mL for every final volume amount of beads desired. Final dilutions of beads will vary depending on initial concentrations of bead solids.

Other concentrations of “buffer to bead” may be utilized. The actual concentrations used will be determined by the particular parameters of test. However, it should be noted that centrifuge containers having conical bottoms potentially increase the clumping of the beads during the centrifuge procedure. Therefore, flat bottomed containers should preferably be used to reduce or eliminate this clumping. Further, volumes may change with each new lot of beads, antigen, conjugate or buffer.

In a preferred embodiment of the invention, the surfactant amount, or the amount of chemical stabilizers used by the bead manufacturer to prevent clumping, within the stock solution should not exceed 5% in the initial (stock) beads. Increases in the surfactant, which are the chemicals that maintain or facilitate single bead distribution while being stored at the manufacturer, make washing difficult. This in turn makes it more difficult to coat beads with antigens. The invention therefore preferably requires that the degree of surfactancy be kept within acceptable limits or tolerances in order to ensure proper attachment of the antigen or other substances to the beads. This ultimately facilitates use of the bead system in actual testing conditions in a manner which will allow for reduced or no wash procedures.

It is important to bear in mind that variations in bead size may occur between lots of the various sized beads. Therefore, it is also necessary to check the accuracy of the lots so that the variations are within acceptable tolerances prior to coating of the beads with the antigen. Moreover, each bead lot should be washed twice before coating, run on a flow cytometer, and the coefficient of variation checked for acceptability.

At this point, size specific amounts of beads are added to each of the test containers. The amount of beads added to each test container will depend upon the volume required, and the amount of buffer therein, as described above.

### EXAMPLE

If the desired final kit volume of RnP/Sm coated beads is 24 mLs, or an amount required for about 240 tests, then 1 mL of neat, manufactured beads is equivalent to 16 mLs of kit beads. Therefore, 24 mLs of the desired beads divided by 16 mLs of the kit beads indicates that 1.5 mLs of neat manufactured product are required. Thus, and with reference to the Table I above, it should be noted that 1.5 mLs of buffer plus 150 uL of 4μ beads would be required to prepare the test sample of RnP/Sm-4μ antigen-bead complex.

Using similar calculations, the amount of any size specific bead can be calculated, and with the above formula, the remaining five beads are added to the remaining five test containers containing the five different antigens and buffer, as already described above.

Each container is then vortexed until a uniform suspension of beads is obtained, and an appropriate amount of antigen is thereafter thawed which would be necessary to obtain the desired final concentration of beads. Each bead suspension is carefully centrifuged and the final supernatant removed. The bead pellet is vortexed until it is evenly distributed. Antigen is applied directly to the bead pellet, vortexed and then resuspended in the calculated, undiluted volume and left to incubate. Table II below shows the recommended amount of antigen per 1 mL of undiluted bead buffer solution.

TABLE II

| | Amount units |
|---|---|
| Antigen | (μL)/mL beads |
| Scl-70 | 50 |
| RnP/Sm | 40 |
| SS-A | 40 |
| SS-B | 40 |
| dsDNA | 10 |
| SM | 20 |

### EXAMPLE

Assume for the sake of this example that RnP/Sm beads indicated above are to be used, then 1.5 mLs of manufactured beads are to be made. In this instance, Table II indicates that for every 1 mL of beads, 40 units (μLs) of antigen are to be added to the bead/buffer suspension. Therefore, at 1.5 mLs total beads/buffer volume, 40×1.5=60 units (μLs) of antigen are to be added to the solution.

It is important to bear in mind that the quantity of antigen may vary slightly from one lot to another. Therefore, it is advisable to titer all new lots. The RnP/Sm antigen, particularly, tends to become unstable after two years, even if frozen at −80° C., and, in any event, to ensure the continued integrity of the results of the assay, storage time should be carefully monitored and controlled. Failure to monitor and control these factors may cause antigenicity to change in a negative way, since coated antibodies may no longer recognize the antigen on the beads.

In carrying out the centrifuge process, it is also important that conical centrifuge tubes be avoided if at all possible since the concentration effect at the bottom of these tubes may, once more, cause bead clumping, and these may ultimately be seen on the flow cytometer as doublets, which may be defined as two or more beads of the same size forming matrices and visually seen 2×, 4×, etc. the size of the original bead. Thus, flat-bottom tubes or containers are preferred at all times, as indicted above.

Upon completion of the centrifugation process, the beads are removed from the centrifuge, and the supernatant is carefully decanted or aspirated. Care should be taken not to discard the beads at this time. The beads are then resuspended by vortexing, which continues as necessary, and preferably until all the material is no longer in a “pellet” form (“pellet” being defined as a concentrated layer of beads formed by centrifugation).

At this time, more buffer is added to each of the bead containers, and the volumes of buffer added should preferably be equal to the initial calculated amounts added, as described above with respect to the calculation of the amounts of beads which are added to the buffer solution. For example, with respect to the RnP/Sm example above, it will be necessary to resuspend in 1.5 mLs s of buffer.

At this point in the preparation of the antigen bead coating process, the centrifuging for 10 minutes at 1400 g in a refrigerated centrifuge is repeated, followed by removal of the beads from the centrifuge and carefully decanting or aspirating the supernatant. Further, the beads are resuspended by vortexing, again, until all bead material is no longer in the “pellet” form.

These various prewashing steps are very important and significant to the invention, since the prewashing removes surfactants and helps to prevent the clumping of the beads during the assay and flow cytometry procedures.

Upon completion of the prewashing, the specified amount of antigen is added directly to each of the washed beads, prior to adding volume of buffer. Addition of antigen directly to the beads also ensures a uniform coating. Binding of proteins, in a passive system, occurs within a matter of minutes. The mixture is vortexed, and a specific amount of buffer is added to each container in a manner along the lines set forth above. Further gentle vortexing takes place. Concentrating beads in smaller initial volumes of buffer allows for equal distribution of the antigen on the bead surface.

Once the appropriate amounts of antigen have been added to the beads, the bead suspensions are incubated at 2-8° C. for from several minutes to hours. After incubation, they are centrifuged for about 10 minutes at about 1400 g in a refrigerated centrifuge. The coolness provided by the refrigeration of the centrifuge helps prevent clumping of the beads, which will not pack as significantly as they would at room temperature.

The product is then incubated for approximately 12-18 hours at 4° C., after which the bead/antigen solution is gently resuspended. Further washing preferably takes place at this, point by centrifuging the bead suspension, removing the beads, and decanting or aspirating the supernatant. Most bead suspensions (except dsDNA) require an additional wash with a 0.5% BSA in carbonate buffer to eliminate non-specific binding sites. Centrifugation and decanting will follow.

Buffer is then added to the final kit volume which is produced at this point. In the example above, the final kit volume is 16 times the neat manufacturing volume. For example, the original neat manufacturing incubation volume is 1.5 mLs, and the final volume will be 1.5×16 mLs for a 1:16 dilution, to require that 24 mLs of buffer be added. Final volumes may vary depending on the type of kit, the initial concentrations of the beads, loss during manufacturing, and the total number of beads in the assay tube.

The test mixture is then treated by mixing the entire volume until it is evenly resuspended, and each bead/antigen suspension is packaged into appropriate bottles and a kit is formed or created. A kit may contain, for example, 1:20 dilution of conjugate in 5 mL aliquots per 100 test kit. Additionally, the bead components may be packaged separately and later mixed together by the end user. The bead components may alternately be packaged as a mixture or slurry of all the beads in one vial. Positive and negative controls may also be added to the kit to complete the package. Similarly, a sample diluent (0.5% BSA in carbonate buffer with azide) may also be included. The kit package is then subjected to quality control tests, as described below.

Quality Control

Set forth below are quality control steps which should preferably be followed to ensure that the bead product has acceptable levels of quality.

1. New antigen lots produced must be stored at −80° C.

2. All new lots of antigen +/−10 units (μL) should be titered from the current lot.

3. All extra antigen generated should be stored in aliquots at −80° C.

4. The RnP/Sm antigen should not be stored longer than two years at −80° C. due to antigenic changes.

5. All new bead lots must be processed and run on a flow cytometer to confirm C.V.s prior to use.

6. The C.V. percent should be checked to ensure it is within the range of +/−0.5% of the Certificate of Accuracy received from the source or manufacturer of such beads.

7. Most importantly, the beads should be manufactured so as to have no more than 0.5% surfactant, otherwise antigen coating will not occur. This lower surfactant level is highly advisable in order to ensure that the antigens stick to the beads (it is to be noted that the bead and the antigen attach to each other by passive absorption).

8. The undiluted conjugate, or indicator reagent, is stored, preferably at −80° C. in the dark, until ready for use.

9. It is preferred that all new lots of positive controls, made from pools of monospecific human or recombinant products, be titered +/− one dilution of current titer. In this regard, monospecific “gold” standards should be utilized as reference material. These standards have been certified by a group such as the CDC or NIH to perform at a given positivity.

10. The results of the conjugate titer must fall within +/− one dilution of the previous value.

11. Preferably, the target values for the individual beads of the assay should demonstrate C.V.s of approximately 5.0% or less. Separation must be clear and distinct on the forward (size) scatter histogram.

12. Importantly, the carbonate buffers described above, should be made fresh with each new lot of beads, unless used within approximately one week of preparation.

13. The pH of the buffer should be checked so as to ensure that it is preferably 9.6, with a range of +/−0.1.

14. Any bead product which, upon analysis, demonstrates agglutination prior to use, should be discarded, since such a bead product will have less than optimal effect in the assay.

It will be appreciated from the detailed description provided above that specific antigens can be coated to the beads. However, as mentioned above, the antigens indicated in the above example are representative only, and other antigens, antibodies, enzymes, viruses, ligands or other chemicals may be used for the detection of target compositions in conjunction with the beads for flow cytometer assays.

The “no-wash” technical procedure described herein can be used in all the bead assays so that the proper amounts of each bead size and reagents are put into one tube simultaneously, incubated, and then followed up with some kind of fluorescent indicators).

The various bead concentrations provided and indicated in the description above, in the buffer, causes the beads to maintain their uniformity, and to prevent the clumping and the production of doublets. In other words, the bead complexes continue to constitute unique populations for analysis on the flow cytometer. Furthermore, the antigens are coupled to the surface of the beads, and do not cross-react with each other thus preventing doubling. The absence or minimization of cross-reaction may be due to the nature of the antigen itself, as well as the preferred pH of the buffer. Further, relatively little background is produced.

It is emphasized that the selection of the pH buffer is important in creating the bead-antigen assay. Also emphasized is the specific concentration of surfactant, preferably no more than 5%, so that the bead has just sufficient coating to keep it in suspension, and at the same time keep the solid bead mixture in suspension as well. Most beads do indeed have surfactant, and when this exceeds 5%, the beads resist effective washing and coating. It will, of course, be appreciated that the surfactant is eventually washed off prior to coating the beads with the antigen, but an excess of surfactant does increase the difficulty in effective washing steps for removing it.

One of the important procedures in the invention is that, once the surfactant has been washed off, the antigen is added directly to the bead. Antigens could also be covalently coupled or chemically attached to the beads. Thus, if 1 mL of bead solution is provided, it is centrifuged quickly and at high speed to compress the beads to a pellet, whereupon the supernatant is removed, the bead pellet resuspended, and the antigen added directly to the bead pellet. Vortexing then follows, which facilitates an equal coating on the beads, and allows the antigens to absorb passively onto the beads and remain there. Other methodologies where antigen is added to a suspension of beads do not permit as uniform a coating because of chemical or mechanical processes that may be too harsh. The particular procedures described above represent significant advances in order to fine-tune the coating and after-coating procedures to make sure the bead is stable, with the antigen formed on the surface, over a prolonged period of time.

Stability of the product is obtained by storing it in the buffer, as described. The carbonate buffer provides the pH necessary for the proper forces, whether they are absorption or electrostatic, to bind the antigen to the bead. Further, once the bead has been coated with the particular antigen, it is left in the buffer which again stabilizes that bead and maintains the equilibrium of the antigen. Product results in the addition of antigen coated beads into a reaction vial (such as a test tube, microtiles plate, etc.), adding a predetermined amount of sample (for example, serum, whole blood, etc.) and incubating. Without washing the bead sample complex, add the fluorescent indicators) and, once again, incubate. At this point, the sample can be analyzed on the flow cytometer.

The invention is not limited to the precise details and methods as described above. Variations can occur within the scope of the invention.

Some variations are as follows. Kits: Viral/bacterial antibodies or antigens are attached; beads impregnated with fluorescenated dyes and different sizes, quantitation procedures for various antigens on the beads; being able to add more beads to the no-wash system after the analysis has been run (to conserve patient sample and reagents, thus decreasing costs). Modularity of the beads, using one, two or more combinations of beads in one tube; mixing and matching different types of assays (e.g. antibody detection with a antigen detection).

1. A method of making a no wash bead based assay, the method comprising:
preparing a first reagent comprising a buffer;
preparing a second reagent comprising a protein;
preparing beads of preselected size and having a coefficient of variation less than 5%, including washing the beads in the buffer to form a bead-buffer matrix and reducing the surfactancy of the beads to an effective amount;
adding an antigen for detecting the presence of a target species to the bead-buffer matrix such that the antigen attaches to the beads to form a bead-antigen mixture, the surfactancy of the beads facilitating attachment of the antigen thereto;
adding buffer to the bead-antigen mixture and thereafter incubating the mixture; and
adding second reagent to the bead-antigen mixture to reduce or eliminate non-specific binding sites.

2. A method as claimed in claim 1 wherein the first reagent is a carbonate buffer.

3. A method as claimed in claim 2 wherein the carbonate buffer has a pH in the range of 9.0-10.0.

4. A method as claimed in claim 3 wherein the carbonate buffer has a pH of 9.6.

5. A method as claimed in claim 1 wherein the second reagent is bovine serum albumin (BSA).

6. A method as claimed in claim 5 wherein the BSA comprises a 0.1-5.0% BSA in saline.

7. A method as claimed in claim 6 wherein the BSA is a 0.5% BSA in saline.

8. A method as claimed in claim 1 wherein the size of the beads is selected from one or more of the group consisting of 3μ latex beads, 4μ latex beads, 5μ latex beads, 6μ latex beads, 7μ latex beads, 8μ latex beads, 9μ latex beads and 10μ latex beads.

9. A method as claimed in claim 8 wherein the beads are selected so as to have a coefficient of variation not exceeding 5%.

10. A method as claimed in claim 9 wherein the beads are selected so as to have a coefficient of variation not exceeding 1.3%.

11. A method as claimed in claim 8 wherein multiple sizes of beads are selected.

12. A method as claimed in claim 1 wherein the antigens added are selected from the group consisting of RNP/SM, SM, SS-A, SS-B, SCL-70 and dsDNA.

13. A method as claimed in claim 1 wherein the antigens are selected from one of more of the group consisting of histones, lipids, viral antibodies, viral antigens, bacterial antibodies, bacterial antigens, recombinant proteins, and cellular antigens.

14. A method as claimed in claim 1 wherein the surfactancy of the beads is reduced to no more than 5% in order to enhance the ability to coat the beads with antigens.

15. A method as claimed in claim 14 wherein the surfactancy is no more than 0.% of the beads.

16. A method as claimed in claim 1 wherein the bead-based assay is prepared in a flat-bottom container.

17. A method as claimed in claim 1 wherein the bead-buffer matrix is subjected to at least one prewashing step.

18. A method as claimed in claim 1 further comprising the step of centrifuging the bead-buffer matrix and the bead-antigen mixture, and the resuspension thereof.

19. A method as claimed in claim 1 further comprising the step of vortexing the bead-buffer matrix and the bead-antigen mixture, and the resuspension thereof.

20. A method of manufacturing a no wash kit for carrying out a bead based assay for testing for the presence of a target substance, the method comprising:
preparing a first reagent comprising a buffer;
preparing a second reagent comprising a protein;
preparing a third reagent comprising an indicator antibody, the third reagent being selected for its ability to identify the target substance;
preparing beads of preselected size and having a coefficient of variation less than 5%, including washing the beads in the buffer to form a bead-buffer matrix and reducing the surfactancy of the beads to an effective amount;
adding an antigen for detecting the presence of the target substance to the bead-buffer matrix such that the antigen attaches to the beads to form a bead-antigen mixture, the surfactancy of the beads facilitating attachment of the antigen thereto;
adding buffer to the bead-antigen mixture and thereafter incubating the mixture;
adding second reagent to the bead-antigen mixture to reduce or eliminate non-specific binding sites;
placing the bead-antigen mixture in a first container for use in an assay test procedure; and
placing the third reagent in a second container for use during the test procedure, the third reagent being used after the target substance has attached to the bead for the purpose of identifying the target substance.

21. A method as claimed in claim 20 wherein the third reagent comprises goat anti-human Ig.

22. A method as claimed in claim 20 comprising a plurality of bead-antigen mixtures, wherein a plurality of different antigens are each attached to a bead of different size.

23. A method as claimed in claim 22 wherein the bead sizes are selected from the following: 3μ latex beads, 4μ latex beads, 5μ latex beads, 6μ latex beads, 7μ latex beads, 8μ latex beads, 9μ latex beads and 10μ latex beads.

24. A method as claimed in claim 22 wherein one or more of the antigens are selected from the group consisting of RNP/SM, SM, SS-A, SS-B, SCL-70 and dsDNA.

25. A no wash kit for carrying out a bead based assay for testing for the presence of a target substance, the kit comprising a bead based assay prepared according to the method of claim 1, and a detector assay reagent comprising an indicator antibody, the detector assay reagent being selected for its ability to identify the target substance.

26. A no wash bead based assay for testing for the presence of a target substance, the assay being prepared according to the method of claim 1.
